(19) United States
(12) Patent Application Publication
(10) Pub. No.: US 2023/0181868 A1
(43) Pub. Date: Jun. 15, 2023

(54) SYSTEMS AND METHODS TO PROVIDE A RELAXATION REGIMEN TO AN OCCUPANT OF A VEHICLE

(71) Applicant: Ford Global Technologies, LLC, Dearborn, MI (US)

(72) Inventors: Kwaku Prakah-Asante, Commerce Twp., MI (US); Ayush Shah, Belleville, MI (US)

(73) Assignee: Ford Global Technologies, LLC, Dearborn, MI (US)

(21) Appl. No.: 17/546,933

(22) Filed: Dec. 9, 2021

(51) Int. Cl. A61M 21/02; B60N 2/00; B60N 2/90; B60R 16/037; B60Q 9/00

(52) U.S. Cl. CPC A61M 21/02; B60N 2/002; B60N 2/976; B60R 16/037; B60Q 9/00; A61M 2021/0022

(57) ABSTRACT

The disclosure is generally directed to providing a relaxation regimen to an occupant of a vehicle. In one example method, a processor evaluates a physiological condition of the occupant. The physiological condition can be a heart rate, hear-rate variability, a breathing rate, and/or a blood oxygen level. The processor also identifies an opportune time to offer the relaxation regimen. The relaxation regimen may be offered based on evaluating the physiological condition of the occupant and on identifying the opportune time. Furthermore, the relaxation regimen may be recommended based on the customer preference or usage patterns and the opportune time. The opportune time can be when the vehicle is moving in a cruising mode of operation, is in a stopped or parked state, or is moving in stop-and-go traffic, or location. In another example method, a processor may offer the relaxation regime based on a movement status of the vehicle.

### BACKGROUND

Customer experience and relaxation, may be-promoted in various ways, such as by playing music on an audio system in the vehicle, listening to an audio book, or conversing with passengers in the vehicle. It is therefore desirable to provide a solution that not only provides relaxing experience measures but also does so at appropriate times in an appropriate manner.

### BRIEF DESCRIPTION OF THE DRAWINGS

A detailed description is set forth below with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 illustrates an example vehicle that includes a relaxation regimen provider system in a vehicle in accordance with an embodiment of the disclosure.

FIG. 2 illustrates some example functional blocks of a relaxation regimen provider system in a vehicle in accordance with an embodiment of the disclosure.

FIG. 3 shows a first example display on a display screen in accordance with the disclosure.

FIG. 4 shows a second example display on a display screen in accordance with the disclosure.

FIG. 5 shows two example graphs of parameters associated with a relaxation regimen provider system in a vehicle in accordance with an embodiment of the disclosure.

FIG. 6 shows an example route map with high driving demand activity inducing sections that may be identified by a relaxation regimen provider system in order to offer a relaxation regimen in accordance with an embodiment of the disclosure.

FIG. 7 shows an example graph associated with a recursive computation procedure executed by a relaxation regimen provider system in accordance with an embodiment of the disclosure

FIG. 8 shows an example graph associated with another recursive computation procedure executed by a relaxation regimen provider system in accordance with an embodiment of the disclosure.

FIG. 9 shows a sequence diagram that illustrates various operations associated with providing a relaxation regimen in accordance with the disclosure.

### DETAILED DESCRIPTION

### Overview

In terms of a general overview, embodiments described in this disclosure are directed to systems and methods for providing a relaxation regimen to an occupant of a vehicle. In an example method, a processor evaluates a physiological condition of an occupant of a vehicle (a driver, for example). The physiological condition can be, for example, a heart rate, heart-rate variability, a breathing rate, or a blood oxygen level. The processor also identifies an opportune time to offer the relaxation regimen (a breathing exercise and/or a massage, for example). The relaxation regimen may be offered based on evaluating the physiological condition and on identifying the opportune time. The relaxation regimen may also be recommended based on the customer preference or usage patterns and the opportune time. The opportune time can be, for example, when the vehicle is moving in a cruising mode of operation, is in a stopped state, or is moving in stop-and-go traffic. The opportune time may be identified based on factors such as, for example, a speed of movement of the vehicle, a stopped condition of the vehicle, an acceleration characteristic of the vehicle, a braking characteristic of the vehicle, a direction of travel of the vehicle, a posted speed limit on a road, preferences of the occupant, relaxation regime usage patterns, and/or a location of the vehicle. In another example method, a processor may offer the relaxation regimen based on a driver behavior activity and movement status of the vehicle (speed, acceleration, braking, steering wheel, etc.) and an opportune time to offer the relaxation regime, such as, for example, when the vehicle is moving in a cruise control mode of operation, is in a stopped state, or is moving in stop-and-go traffic.

### ILLUSTRATIVE EMBODIMENTS

The disclosure will be described more fully hereinafter with reference to the accompanying drawings, in which example embodiments of the disclosure are shown. This disclosure may, however, be embodied in many different forms and should not be construed as limited to the example embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described example embodiments but should be defined only in accordance with the following claims and their equivalents. The description below has been presented for the purposes of illustration and is not intended to be exhaustive or to be limited to the precise form disclosed. It should be understood that alternate implementations may be used in any combination desired to form additional hybrid implementations of the present disclosure. For example, any of the functionality described with respect to a particular device or component may be performed by another device or component. Furthermore, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments.

Certain words and phrases are used herein solely for convenience and such words and terms should be interpreted as referring to various objects and actions that are generally understood in various forms and equivalencies by persons of ordinary skill in the art. For example, the word “sensor” may be used interchangeably with the word “detector.” Either word as used in this disclosure refers to various devices, such as, for example, an ultrasonic sensor, a radar detector, and an imaging device, such as a camera that is used to capture an image (or a video clip) of the driver of a vehicle, for example, for evaluation by a processor. The word “data” may be used interchangeably with the word “information.” Either word pertains to any of various forms of input to a processor. The word “condition” may be used interchangeably with words, such as “situation,” “state,” and “scenario.” It should be understood that a “driver” is one “occupant” of a vehicle and that one or more passengers of the vehicle are also “occupants” of the vehicle. As such, various items referred to herein with respect a “driver” must be interpreted in context as being applicable to a passenger of the vehicle as well. It should be further understood that the word “example” as used herein is intended to be non-exclusionary and non-limiting in nature.

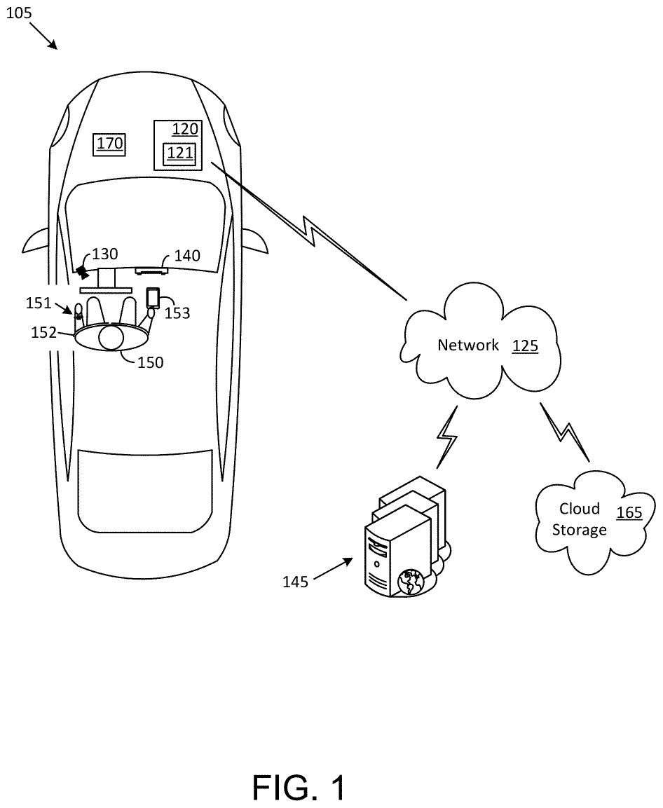

FIG. 1 illustrates an example vehicle 105 that includes a relaxation regimen provider system 120 in accordance with an embodiment of the disclosure. The vehicle 105 may be any of various types of vehicles such as, for example, a gasoline powered vehicle, an electric vehicle, a hybrid electric vehicle, or an autonomous vehicle, and includes various types of sensors and detectors configured to provide various functionalities.

For example, a first functionality provided by a sensor can pertain to monitoring a physiological condition of the driver 150 and/or other occupants of the vehicle 105 (heart rate, breathing rate, blood oxygen level, etc.). A second functionality provided by a sensor can pertain to characterizing various driver activity and actions performed by the vehicle 105 (speed, acceleration, braking, cruise control, etc.).

A few example sensors that provide the first functionality can include a camera 130 mounted inside the vehicle 105, a pressure transducer mounted upon a steering wheel of the vehicle 105, and an infrared temperature sensor mounted inside the vehicle 105. The camera may be mounted upon the front windshield of the vehicle 105 and arranged to capture images of the driver 150 and/or other occupants of the vehicle 105. The images may be conveyed to the relaxation regimen provider system 120 and evaluated by a processor 121 of the relaxation regimen provider system 120 to detect conditions such as, for example, stress that may be visible on the face of the driver 150. The images may be provided in the form of digital images, video clips, and/or real-time video streams. The pressure transducer mounted upon the steering wheel of the vehicle 105, may produce sensor signals that indicate an amount of pressure exerted by a grip of the driver 150 upon the steering wheel. The sensor signals may be conveyed to the relaxation regimen provider system 120, which evaluates the sensor signals in order to determine an amount of stress that may be present in the driver 150. The infrared temperature sensor can provide to the relaxation regimen provider system 120, temperature measurements of the driver 150. The temperature measurements may indicate a physical state of the driver 150.

In some scenarios, the sensors that provide the first functionality may be brought into the vehicle 105 by one or more occupants of the vehicle 105. For example, the driver 150 may bring into the vehicle 105, a wearable device 151, a smart wearable item of clothing 152, a smartwatch, and/or a smartphone 153. Such devices may be configured to automatically establish wireless communications with the relaxation regimen provider system 120 via communication technologies such as Bluetooth®, cellular, near-field communication (NFC), Wi-Fi, Wi-Fi direct, machine-to-machine communication, and/or man-to-machine communication.

The wearable device 151 can, for example, include a fitness tracker and/or an activity tracker that monitors and provides to the relaxation regimen provider system 120, various physiological parameters associated with the driver 150. Example physiological parameters can include but not limited to heart rate, heart rate variability, breathing rate, and blood oxygen level. The smart wearable item of clothing 152 and the smartphone 153 may provide to the relaxation regimen provider system 120, various physiological parameters associated with the driver 150 such as those described above with reference to the wearable device 151.

A few example sensors that provide the second functionality is a speed sensor, a brake sensor, a fuel consumption sensor, and various pressure transducers. These sensors may be configured to provide to the relaxation regimen provider system 120, data that can be evaluated by the relaxation regimen provider system 120 to characterize various actions performed by the vehicle 105. In one implementation, the sensors provide the data directly to the relaxation regimen provider system 120. In another implementation, a vehicle computer 170 may be configured to provide various types of information to the relaxation regimen provider system 120.

The vehicle computer 170 may provide the information either in an unprocessed format or in a processed format. The processed format may be produced by the vehicle computer 170 based on evaluating sensor data from one or more sensors and translating the sensor data to information such as, for example, an acceleration characteristic of the driver of the vehicle 105, a braking characteristic of the vehicle 105, a cruise control characteristic of the vehicle 105. The vehicle computer 170 may also provide to the relaxation regimen provider system 120, information pertaining to traffic around the vehicle 105. In an example scenario, the information provided by the vehicle computer 170 may indicate to the relaxation regimen provider system 120 that the vehicle 105 is moving in stop-and-go traffic.

The vehicle computer 170 may also provide some other types of information to the relaxation regimen provider system 120, such as certain actions and behavior performed by the driver 150. For example, the vehicle computer 170 may monitor a fuel injection system and/or a braking system of the vehicle 105 to determine that the driver 150 is executing an acceleration operation or a braking operation upon the vehicle 105.

The vehicle computer 170 may not only be communicatively coupled to the relaxation regimen provider system 120 but also to other elements in the vehicle 105 such as, for example, an infotainment system 140. The infotainment system 140 may include a combination of various entertainment devices (such as a radio, streaming audio solutions, and USB access ports for digital audio devices) with elements, such as a navigation system that provides navigation instructions and maps upon a display screen of the infotainment system 140. A software application may be downloaded into the infotainment system 140 and the vehicle computer 170 (as well as into devices brought into the vehicle 105, such as, for example, the smartphone 153) in order to execute various operations in accordance with the disclosure.

The infotainment system 140 can also include a human machine interface (HMI) that allows an occupant of the vehicle 105 to communicate with the relaxation regimen provider system 120. In an example scenario, the relaxation regimen provider system 120 may display on the HMI, a request to initiate a relaxation regimen. The driver 150 (or another occupant of the vehicle 105) may accept the request. One example relaxation regimen, is a breathing exercise, wherein the relaxation regimen provider system 120 provides instructions, guidance, and tips via the HMI. Another example relaxation regimen is a massage therapy session. In this case, the relaxation regimen provider system 120 may transmit signals (control signal, trigger signal, etc.) to a massager that may be built into a seat of the driver 150. The driver 150 may provide input regarding the massage therapy session to the relaxation regimen provider system 120 via the HMI.

The relaxation regimen provider system 120 may also include a wireless communication system that allows the relaxation regimen provider system 120 to communicate with various devices. In the illustrated example, the relaxation regimen provider system 120 is communicatively coupled via a network 125 to a server computer 145 and cloud storage 165.

The network 125 may include any one, or a combination of networks, such as a local area network (LAN), a wide area network (WAN), a telephone network, a cellular network, a cable network, a wireless network, and/or private/public networks, such as the Internet. For example, the network 125 may support communication technologies, such as TCP/IP, Bluetooth, cellular, near-field communication (NFC), Wi-Fi, Wi-Fi direct, machine-to-machine communication, and/or man-to-machine communication.

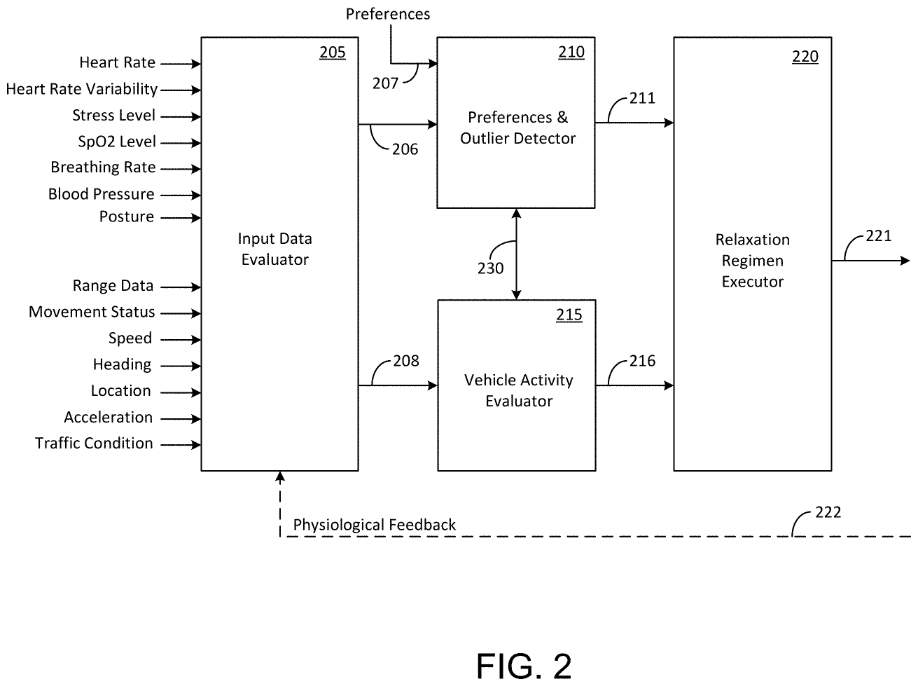

FIG. 2 illustrates some example functional blocks of the relaxation regimen provider system 120 in accordance with an embodiment of the disclosure. Some portions of the functional blocks can be implemented in hardware and other portions in software. The software portions can be provided in the form of computer-executable instructions that are stored in a memory and are executed by a processor for performing various operations in accordance with the disclosure. In an example implementation, the aforesaid computer-executable instructions are stored in a memory (not shown) provided in the relaxation regimen provider system 120 and can be executed by the processor 121 for performing various operations in accordance with the disclosure. In another example implementation, some or all of the aforesaid computer-executable instructions are stored in cloud storage 165 and/or in a memory of the server computer 145. The computer-executable instructions may be retrieved and executed by a processor in the server computer 145 and/or by the processor 121 in the relaxation regimen provider system 120 for performing various operations in accordance with the disclosure.

The example functional blocks include an input data evaluator 205. The input provided to the input data evaluator 205 may be broadly categorized into two categories. The first category is data associated with an occupant of the vehicle 105 (the driver 150, for example) and the second category is data associated with the vehicle 105 and the environment. Data in both categories can vary over time.

The illustrated example data associated with the occupant of the vehicle 105 contains information pertaining to a physiological condition of the driver 150. The physiological condition of the driver 150 can include, for example, heart rate, heart rate variability, stress level, blood oxygen level (indicated as a SpO2 level), breathing rate, blood pressure, and posture. This data can be provided to the input data evaluator 205 by various sources such as, for example, sensors in the vehicle 105 (camera 130, transducer in steering wheel, etc.) and devices brought into the vehicle 105 by the driver 150 (wearable device 151, smart wearable item of clothing 152, smartphone 153, etc.).

As indicated above, data associated with the driver 150 can vary over time. For example, a heart rate of the driver 150 at a first instant in time when an accident was averted, for example, can be high in comparison to a second instant in time when the vehicle 105 is traveling under cruise control. As another example, the blood pressure of the driver 150 at a first instant in time when the driver 150 is stressed can be high in comparison to a second instant in time when the driver 150 has executed a relaxation regimen offered by the relaxation regimen provider system 120. The heart-rate of the driver 150 at the second instant in time is one example of various types of physiological data that can be provided to the relaxation regimen provider system 120 in the form of feedback (indicated by a dashed line 222). The feedback may be evaluated by the relaxation regimen provider system 120 in order to determine an effectiveness of a relaxation regime provided to the driver 150 by the relaxation regimen provider system 120. In some cases, the effectiveness may be quantified by an amount of reduction in blood pressure, breathing rate, and/or heart rate, for example.

The illustrated example data associated with the vehicle 105 provides information pertaining to various functional, operational, and situational conditions of the vehicle 105 at various times. Some examples of data associated with the vehicle 105 can include, for example, range data, movement status, speed, steering heading angle, location, acceleration, and traffic conditions around the vehicle 105. The data can be provided to the input data evaluator 205 by various sources such as, for example, sensors in the vehicle 105 (speed sensor, accelerometer, location sensor, etc.) and/or the vehicle computer 170.

The data associated with the vehicle 105 can vary over time. For example, a speed of the vehicle 105 at a first instant in time when the driver 150 is in control can be different than at a second instant in time when the vehicle 105 has been placed in a cruise control mode of operation. As another example, a speed of the vehicle 105 at a first instant in time when the driver 150 is in control will be higher than at a second instant in time when the vehicle 105 has stopped at a traffic light.

The input data evaluator 205 may produce a first output (illustrated by a line 206) associated with various physiological conditions of the driver 150 and a second output (illustrated by a line 208) associated with the vehicle 105. The first output that is associated with the driver 150 is provided to a preferences and outlier detector 210. The preferences and outlier detector 210 evaluates the various physiological conditions of the driver 150 of the vehicle 105 to detect outlier conditions. In an example implementation, an outlier condition may be detected by comparing a physiological condition provided on line 206 (a heart rate of the driver 150, for example) against a nominal threshold value (a nominal heart rate, for example). In one case, the nominal threshold value may be an average heart rate of the driver 150 measured over a period of time. In some applications, the nominal heart rate may be provided as input by the driver. Yet in some applications, the average heart rate may be calculated using various techniques such as, for example, processor-executed learning procedures, artificial intelligence, historical data evaluation, etc.

The preferences and outlier detector 210 may also be provided an additional input (illustrated by a line 207) associated with one or more preferences of the driver 150. In an example implementation, the driver 150 may provide a preference that indicates a vehicle operating state during which the driver 150 prefers to be offered a relaxation regimen (a breathing exercise, for example). Accordingly, the relaxation regime may be offered over a period of time when, for example, the vehicle 105 is moving under cruise control, is in a stopped state, or is moving in stop-and-go traffic. Another preference may pertain to selecting a mode of initiation of a relaxation regimen. In one scenario, the mode of initiation may be based on a response provided by the driver 150 to a query posed by the relaxation regimen provider system 120 upon the HMI of the infotainment system 140. In another scenario, the relaxation regimen provider system 120 may automatically initiate an exercise regimen based on evaluating physiological conditions of the driver 150, or an opportune time to offer a relaxation regimen based on the driver preferences.

In an example implementation, the preferences and outlier detector 210 may apply certain decision rules to determine a user preference and/or for outlier detection. An example rule may be defined by the following expression:

If(bio_ind>wᵢor bio_ind≤xᵢ) and u_prefᵢ=vᵢ,then Out_detec=mᵢ

where bio_ind represents a physiological condition detected by a device such as, for example, the wearable device 151, the smart wearable item of clothing 152, or the smartphone 153; xᵢ and wᵢ are user input thresholds or adjusted from real-time data excursions; u_prefᵢ is a user preference for experience; Out_detect: mᵢ=(1−detected, 0); and vᵢ is an action performed by the driver 150 for communication and/or interaction with the relaxation regimen provider system 120 as a part of a relaxation regimen.

The preferences and outlier detector 210 provides an output (illustrated by a line 211) that is coupled into a relaxation regimen executor 220. Various features and operations of the relaxation regimen executor 220 are described below.

The second output produced by the input data evaluator 205 is provided to a driver behavior and vehicle activity evaluator 215 as illustrated by line 208 in FIG. 2. The second output produced by the input data evaluator 205 is data based on evaluation of various functional, operational, and situational conditions of the vehicle 105 at various times. Some examples of such data can include, for example, range data, movement status, speed, steering heading angle, location, acceleration, and traffic conditions around the vehicle 105. The data can be provided to the input data evaluator 205 by various sources such as, for example, by sensors in the vehicle 105 (speed sensor, accelerometer, location sensor, etc.) and/or by the vehicle computer 170.

The driver behavior and vehicle activity evaluator 215 evaluates the information provided by the input data evaluator 205 to detect outlier conditions in the vehicle data. In some cases, the driver behavior and vehicle activity evaluator 215 may interactively communicate with the preferences and outlier detector 210 (illustrated by line 230) to perform some operations.

In an example implementation, an outlier condition may be detected by comparing a vehicle condition provided on line 208 (a speed of the vehicle 105 over a period of time, for example) against a threshold speed value. The threshold speed value, may, for example, correspond to a posted speed limit on a road in which the vehicle 105 is traveling. The comparison may provide various types of information, such as, for example, whether the vehicle 105 is traveling under cruise control, changing lanes, making a turn, is stopped, or is moving in stop-and-go traffic. Other example conditions that may be identified by the driver behavior and vehicle activity evaluator 215 can include, for example, acceleration, steering wheel angle, heading-angle, distance to preceding vehicle, relative velocity, speed, and/or location of the vehicle 105.

Recursive computation and learning of variability and outlier indicators can provide information on a real-time basis in some cases. Such procedures can be used to automatically detect trends of departure from a driver norm behavior and can predict potential significant changes. Data pipeline inputs including acceleration, steering wheel angle, heading-angle, speed, and location. In an example implementation, the vehicle activity evaluator 215 may determine a norm value of an input variable (speed, acceleration, etc.) by a recursive computation procedure that can be expressed as follows:

x̅k=αx̅ₖ₋₁+(1−α)xₖ

where x̅ represents a characterized input variable (acceleration, speed, etc.), a is a constant ranging from 0 to 1 and adapted to the context and activity of the variable. The deviation from the norm is computed as follows:

pₖ=α₂Pₖ₋₁+(1−α₂)(xₖ−x̅ₖ)(xₖ−x̅ₖ)

where p is a deviation from a norm.;

The output of the recursive computation procedure can be scaled as follows:

$$\text{?}$$

$$\text{?} \text{indicates text missing or illegible when filed}$$

where β is a scaling constant, and DV is a deviation value that ranges from 0 to 1.

Multiple input variables may be aggregated as follows:

$$\text{?}$$

$$\text{?} \text{indicates text missing or illegible when filed}$$

where w represents a weight and p is a deviation value.

The driver behavior and vehicle activity evaluator 215 produces an output that is provided to a relaxation regimen executor 220 (illustrated by a line 216 in FIG. 2) together with the output produced by the preferences and outlier detector 210 (illustrated by line 211 in FIG. 2). The relaxation regimen executor 220 identifies a relaxation regimen (breathing exercise, massage, etc.) based on evaluating the output of the driver behavior and vehicle activity evaluator 215 and/or the output of the preferences and outlier detector 210.

In an example implementation, the relaxation regimen executor 220 identifies a relaxation regimen based on application of the following rule:

If(scen_1=sᵢand InN=xᵢ) and u_prefᵢ=Commᵢ then IDM_out=engᵢ

where scen_1 represents a detected operational condition of the vehicle 105. The operational state can be any of various states (represented as i states, where, for example, i=1 can represent a stopped state of the vehicle 105, i=2 can represent a cruise control operating state of the vehicle 105, and i=3 can represent a stop-and-go traffic state). The term InN represents an intelligent notification status, xᵢ can represent one of an “engage” state or a “do not engage” state provided by the driver behavior and vehicle activity evaluator 215. The term u_prefᵢ can represent a preference by the driver 150 such as, for example, a preferred mode of communication for executing a relaxation regimen. The term Commᵢ can represent a multi-modal communication type to be used (via the infotainment system 140, for example) and IDM_out=engᵢ represents an engagement status (1 for recommend, 0 for no engagement, and 2 for automatic engagement, for example) of the relaxation regimen executor 220 with the driver 150.

The output of the relaxation regimen executor 220 (illustrated by line 221) can be provided to various devices in the vehicle 105. In an example implementation, the output can be instructions that are provide via the infotainment system 140 for executing a breathing regimen. In another example implementation, the output can be signals that are coupled into a massaging device (a seat massager, for example) for executing a massage therapy session.

Physiological data associated with the driver 150 can vary between a first time when the output of the relaxation regimen executor 220 (illustrated by line 221) is produced and conveyed to various devices in the vehicle 105 and/or provided in the form of instructions (breathing instructions, for example) and a second instant in time when the relaxation regimen has taken effect (lowering of a breathing rate, increase in blood oxygen (SpO2) level, etc.).

In one implementation, the physiological data at the second instant in time can be provided to the relaxation regimen provider system 120 in the form of feedback (indicated by a dashed line 222). In another implementation, the physiological data of the driver 150 can be fed back into the relaxation regimen provider system 120 in a continuous stream (real-time feedback) so as to allow the relaxation regimen executor 220 to modify an exercise regimen for improving an effectiveness of an exercise regimen.

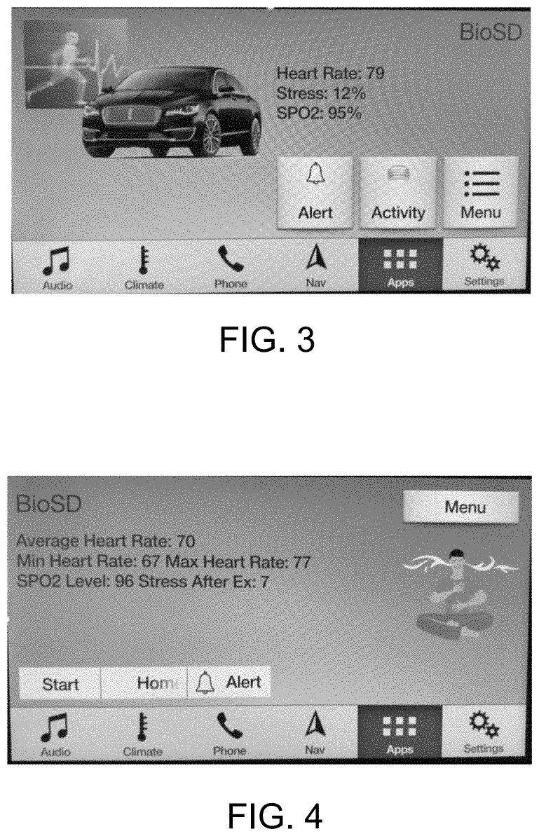

FIG. 3 shows a first example display on a display screen in accordance with the disclosure. In an example implementation, the display screen can be a part of the infotainment system 140. In another example implementation, the display screen can be a part of another device such as, for example, the smartphone 153 of the driver 150. The display indicates some example physiological conditions of the driver 150 prior to commencement of a relaxation regimen provided by the relaxation regimen provider system 120.

FIG. 4 shows a second example display on a display screen in accordance with the disclosure. The display screen can be the one described above with reference to FIG. 3. The second example display indicates some example physiological conditions of the driver 150 after commencement (or after completion) of a relaxation regimen provided by the relaxation regimen provider system 120.

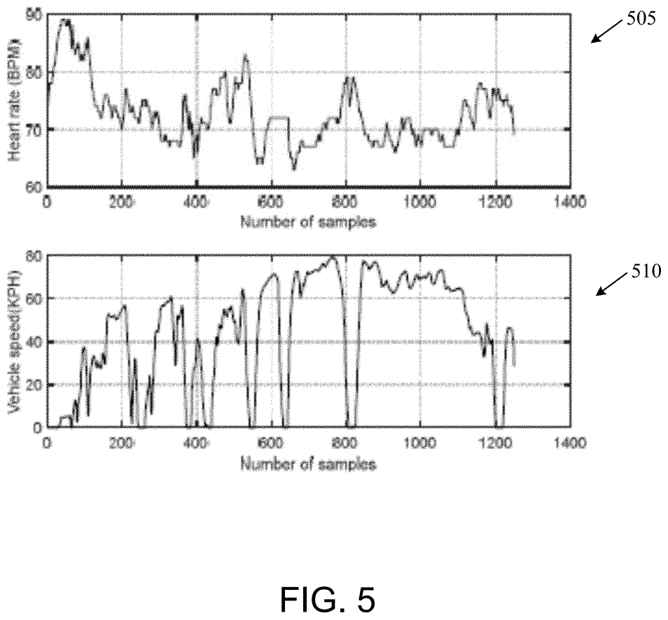

FIG. 5 shows two example graphs of parameters associated with the relaxation regimen provider system 120 in accordance with an embodiment of the disclosure. Graph 505 shows a variation of a physiological condition of the driver 150 over time (a heart rate, in this example). Graph 510 shows a variation of a vehicle movement status of the vehicle 105 over time. The relaxation regimen provider system 120 can evaluate the movement status (a speed of the vehicle 105, in this example) to determine whether the vehicle 105 is in cruising mode undergoing steady speed, is stopped, or is moving in stop-and-go traffic.

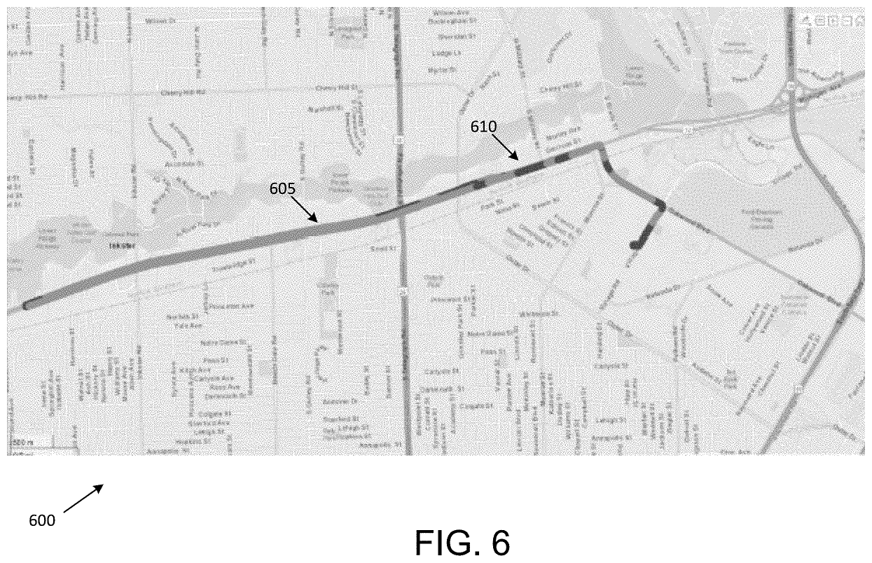

FIG. 6 shows an example route map 600 with high driving demand sections that may be identified by the relaxation regimen provider system 120 in order to offer a relaxation regimen in accordance with an embodiment of the disclosure. The vehicle 105 is traveling at a substantially constant speed over an example section 605 of the route which is relatively free of traffic. Traffic congestion exists in another example section 610 and the vehicle 105 may either stop for a significant amount of time because traffic has ground to a halt or may move in spurts due to a stop-and-go nature of traffic movement.

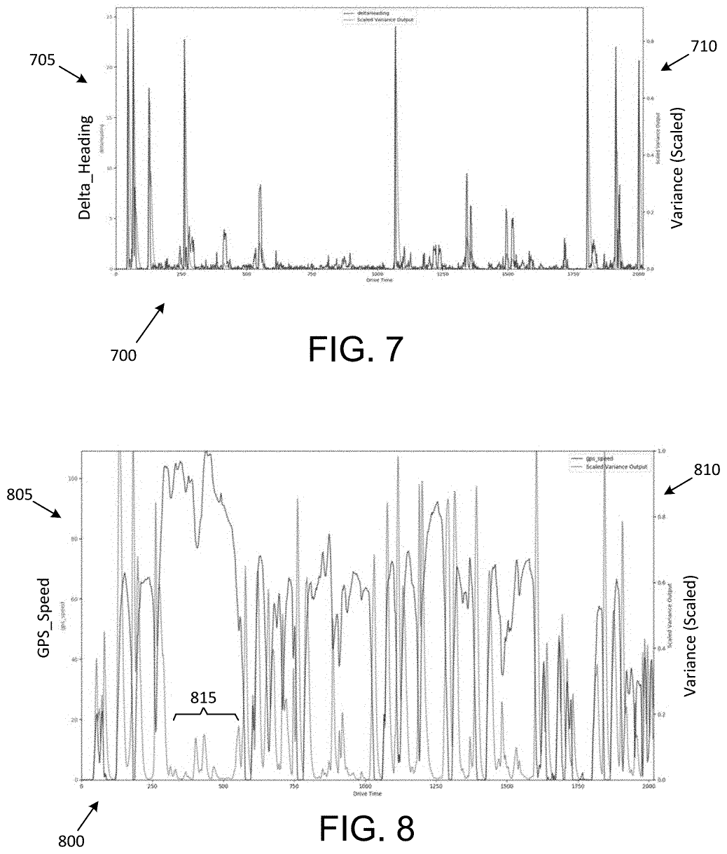

FIG. 7 shows an example graph 700 associated with a recursive computation procedure executed by the relaxation regimen provider system 120 in accordance with an embodiment of the disclosure. The graph 700 has two y-axes. The y-axis 705 indicates delta-heading values (in degrees per second) mapped against x-axis values of drive time (in seconds). The y-axis 710 indicates a scaled variance (normalized from 0 to 1) mapped against x-axis values of drive time (in seconds). A tunable threshold of 0.6 may be used to distinguish between high activity detection and low activity detection. The tunable threshold may be chosen in accordance with a desired level for decision making by the relaxation regimen provider system 120.

FIG. 8 shows an example graph 800 associated with another recursive computation procedure executed by the relaxation regimen provider system 120 in accordance with an embodiment of the disclosure. The graph 800 may be used to identify various locations along a travel route where the vehicle 105 is moving in cruising mode undergoing steady speed. The graph 800 has two y-axes. The y-axis 805 indicates a speed of the vehicle 105. The y-axis 810 indicates a scaled variance (normalized from 0 to 1) mapped against x-axis values of drive time (in seconds). The graph 800 indicates that the speed of the vehicle 105 has a reduced variance between about the 300 second mark and the 550 second mark shown on the x-axis. The reduced variance, which hovers around 0 and is indicated by reference numeral 815, is indicative of the vehicle 105 traveling in cruising mode with reduced speed variance.

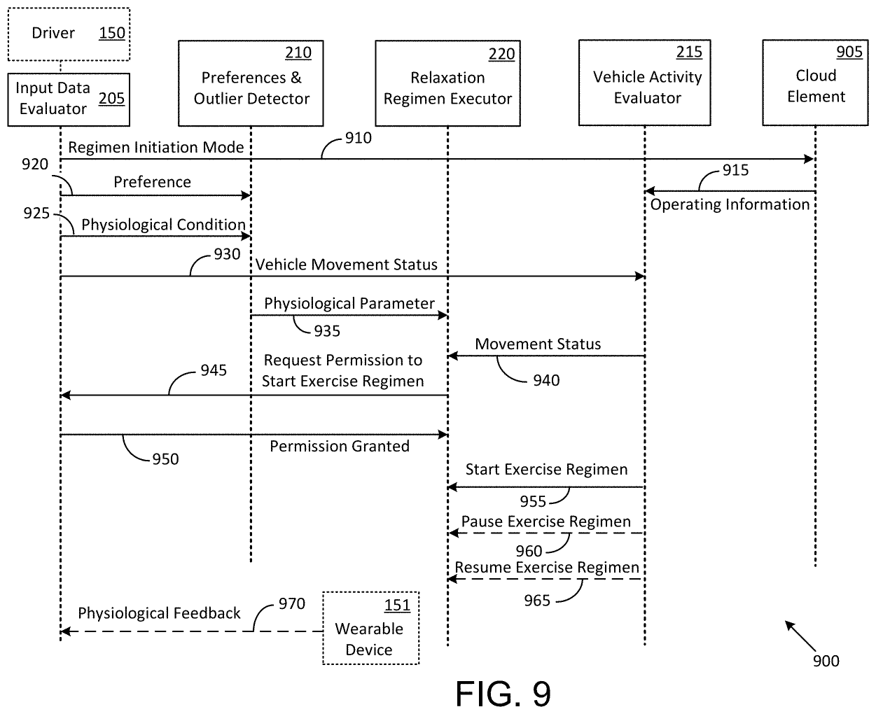

FIG. 9 shows a sequence diagram 900 that illustrates various operations associated with providing a relaxation regimen in accordance with the disclosure. The sequence diagram 900 illustrates a sequence of operations that can be implemented in hardware, software, or a combination thereof. In the context of software, the operations represent computer-executable instructions stored on one or more non-transitory computer-readable media such as a memory device provided in the relaxation regimen provider system 120, that, when executed by one or more processors such as the processor 121 provided in the relaxation regimen provider system 120, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be carried out in a different order, omitted, combined in any order, and/or carried out in parallel. Some or all of the operations described in the sequence diagram 900 may be carried out by the relaxation regimen provider system 120 either independently or in cooperation with other devices such as, for example, the vehicle computer 170, the smartphone 153 of the driver 150, and cloud elements (such as, for example, the server computer 145 and cloud storage 165).

Operation 910 is a relaxation initiation mode selection operation that may be performed by the input data evaluator 205 at a time when the driver 150 starts the vehicle 105. The relaxation initiation mode may be either selected automatically by the relaxation regimen provider system 120 or may be based on input provided to the relaxation regimen provider system 120 by the driver 150. The relaxation initiation mode may provide an indication of an opportune time to initiate a relaxation regimen. The opportune time may be determined on the basis of various factors such as, for example, when a physiological condition of the driver 150 warrants relaxation (high blood pressure, high stress, low blood oxygen level, etc.) and/or when a movement status of the vehicle 105 permits execution of the relaxation regimen (is in cruise control, a stopped condition, or in stop-and-go traffic). It may be undesirable to initiate a relaxation regimen under adverse conditions such as, for example, when the driver 150 is engaged in high driving demand activity or when the driver 150 is operating the vehicle 105 at high speed.

The relaxation initiation mode selection operation may involve communications between the relaxation regimen provider system 120 and a cloud element 905 such as, for example, a server computer. The cloud element 905 may provide information associated with the relaxation initiation mode selection to the vehicle activity evaluator 215 of the relaxation regimen provider system 120 (illustrated as an operation 915).

Operation 920 pertains to the preferences and outlier detector 210 evaluating a preference of the driver 150 provided directly by the driver 150 (indicated by line 207 in FIG. 2) or via the input data evaluator 205.

Operation 925 pertains to the preferences and outlier detector 210 evaluating a physiological condition of the driver 150 (indicated by line 206 in FIG. 2).

Operation 930 pertains to the vehicle activity evaluator 215 evaluating a movement status of the vehicle 105 (as indicated by line 208 in FIG. 2).

Operation 935 pertains to the relaxation regimen executor 220 evaluating an output provided by the preferences and outlier detector 210. The output is associated with a physiological condition of the driver 150 and is indicated by line 211 in FIG. 2.

Operation 940 pertains to the relaxation regimen executor 220 evaluating an output provided by the vehicle activity evaluator 215. The output is indicated by line 216 in FIG. 2. In an example scenario, the output is produced by the vehicle activity evaluator 215 based on evaluating a movement status of the vehicle 105 and/or a physiological condition of the driver 150. The vehicle activity evaluator 215 may evaluate the movement status of the vehicle 105 based on parameters such as, for example, speed characteristics, acceleration characteristics, braking characteristics, vehicle range information, and blind-spot sensor signals and graphs such as the example graphs described above. In an example implementation, evaluating the movement status of the vehicle 105 allows the vehicle activity evaluator 215 to determine, for example, that the vehicle 105 is in an adaptive cruise control mode of operation. In another example implementation, the vehicle activity evaluator 215 may not only evaluate traffic conditions around the vehicle 105 at a current instant in time but may also obtain information to determine an upcoming movement status of the vehicle 105 based on traffic conditions at a later instant in time at a location further along a travel route of the vehicle 105. The traffic condition at the location further along the travel route may be obtained via a device such as a GPS device that may be a part of the infotainment system 140.

Operation 945 pertains to the relaxation regimen executor 220 sending a query to the driver 150 requesting permission to start an exercise regimen. The request may be sent out after the relaxation regimen executor 220 has determined an opportune time to start the relaxation regimen based on evaluating the physiological condition and/or vehicle movement characteristics. In an example implementation, the query may be displayed on a display screen of the infotainment system 140 and/or of the smartphone 153 of the driver 150.

Operation 950 pertains to the permission being granted by the driver 150.

Operation 955 pertains to the start of an exercise regimen (breathing exercise, massage session, etc.) at the opportune time. A change in status of the vehicle movement characteristics may occur subsequently, such as, for example, when the driver 150 disengages cruise control and takes control of the vehicle 105 due to a change in traffic conditions (slowdown as a result of an accident, for example). An ensuing period of time may be inopportune to continue the relaxation regimen. Consequently, operation 960 indicates a pause in the exercise regimen.

Operation 965 pertains to a resuming of the exercise regimen when an opportune time arises once again such as, when the vehicle 105 is placed back in cruise-control mode.

Operation 970 pertains to physiological feedback provided to the input data evaluator 205 (illustrated by dashed line 222 in FIG. 2) by devices such as, for example, the wearable device 151, the smart wearable item of clothing 152, and/or the smartphone 153.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, one skilled in the art will recognize such feature, structure, or characteristic in connection with other embodiments whether or not explicitly described.

Implementations of the systems, apparatuses, devices, and methods disclosed herein may comprise or utilize one or more devices that include hardware, such as, for example, one or more processors and system memory, as discussed herein. An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or any combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of non-transitory computer-readable media.

Computer-executable instructions comprise, for example, instructions and data which, when executed at a processor, cause the processor to perform a certain function or group of functions. The computer-executable instructions may be, for example, binaries, intermediate format instructions, such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

A memory device can include any one memory element or a combination of volatile memory elements (e.g., random access memory (RAM, such as DRAM, SRAM, SDRAM, etc.)) and non-volatile memory elements (e.g., ROM, hard drive, tape, CDROM, etc.). Moreover, the memory device may incorporate electronic, magnetic, optical, and/or other types of storage media. In the context of this document, a “non-transitory computer-readable medium” can be, for example but not limited to, an electronic, magnetic, optical, electromagnetic, infrared, or semiconductor system, apparatus, or device. More specific examples (a non-exhaustive list) of the computer-readable medium would include the following: a portable computer diskette (magnetic), a random-access memory (RAM) (electronic), a read-only memory (ROM) (electronic), an erasable programmable read-only memory (EPROM, EEPROM, or Flash memory) (electronic), and a portable compact disc read-only memory (CD ROM) (optical). Note that the computer-readable medium could even be paper or another suitable medium upon which the program is printed, since the program can be electronically captured, for instance, via optical scanning of the paper or other medium, then compiled, interpreted or otherwise processed in a suitable manner if necessary, and then stored in a computer memory.

Those skilled in the art will appreciate that the present disclosure may be practiced in network computing environments with many types of computer system configurations, including in-dash vehicle computers, personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by any combination of hardwired and wireless data links) through a network, both perform tasks. In a distributed system environment, program modules may be located in both the local and remote memory storage devices.

Further, where appropriate, the functions described herein can be performed in one or more of hardware, software, firmware, digital components, or analog components. For example, one or more application specific integrated circuits (ASICs) can be programmed to carry out one or more of the systems and procedures described herein. Certain terms are used throughout the description, and claims refer to particular system components. As one skilled in the art will appreciate, components may be referred to by different names. This document does not intend to distinguish between components that differ in name, but not function.

It should be noted that the sensor embodiments discussed above may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the present disclosure have been directed to computer program products comprising such logic (e.g., in the form of software) stored on any computer-usable medium. Such software, when executed in one or more data processing devices, causes a device to operate as described herein.

While various embodiments of the present disclosure have been described above, it should be understood that they have been presented by way of example only, and not limitation. It will be apparent to persons skilled in the relevant art that various changes in form and detail can be made therein without departing from the spirit and scope of the present disclosure. Thus, the breadth and scope of the present disclosure should not be limited by any of the above-described example embodiments but should be defined only in accordance with the following claims and their equivalents. The foregoing description has been presented for the purposes of illustration and description. It is not intended to be exhaustive or to limit the present disclosure to the precise form disclosed. Many modifications and variations are possible in light of the above teaching. Further, it should be noted that any or all of the aforementioned alternate implementations may be used in any combination desired to form additional hybrid implementations of the present disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey the information that certain embodiments could include, while other embodiments may not include, certain features, elements, and/or steps. Thus, such conditional language is not generally intended to imply that features, elements, and/or steps are in any way required for one or more embodiments.

That which is claimed is:

1. A method comprising:
evaluating, by a processor, a physiological condition of an occupant of a vehicle;
identifying, by the processor, an opportune time to offer a relaxation regimen to the occupant of the vehicle; and
offering, by the processor and based on evaluating the physiological condition and/or on identifying the opportune time, the relaxation regimen.

2. The method of claim 1, wherein the physiological condition of the occupant comprises one of a heart rate, heart-rate variability, a breathing rate, and a blood oxygen level and wherein the opportune time occurs during one of when the vehicle is moving in a cruising mode of operation, is in a stopped or parked state, or is moving in stop-and-go traffic.

3. The method of claim 1, wherein the occupant is a driver of the vehicle, and wherein identifying the opportune time comprises:
identifying, by the processor, over a period of time, a speed of movement of the vehicle, a stopped or parked condition of the vehicle, an acceleration characteristic of the vehicle, a braking characteristic of the vehicle, a direction of travel of the vehicle, a posted speed limit on a road, preferences of the occupant, relaxation regime usage patterns, and/or a location of the vehicle.

4. The method of claim 1, wherein the opportune time occurs when the vehicle is one of moving in a cruising mode of operation, is in a stopped or parked state, or is moving in stop-and-go traffic and recommending a relaxation regime based on the opportune time.

5. The method of claim 1, wherein the relaxation regimen comprises a breathing exercise and/or a massaging function, and the method further comprises:
tailoring, by the processor, the relaxation regimen based on a preference of the occupant.

6. The method of claim 1, further comprising:
detecting, by the processor, an outlier in physiological data obtained by monitoring the occupant; and
tailoring, by the processor, the relaxation regimen based on addressing the outlier.

7. A method comprising:
monitoring, by a processor, a movement status of a vehicle;
identifying, by the processor, based on monitoring the movement status of the vehicle, an opportune time to offer a relaxation regimen to an occupant of the vehicle; and
offering, by the processor, the relaxation regimen to the occupant of the vehicle at the opportune time.

8. The method of claim 7, wherein monitoring the movement status of the vehicle comprises monitoring a speed of movement of the vehicle, a stopped or parked state of the vehicle, an acceleration characteristic of the vehicle, a braking characteristic of the vehicle, a direction of travel of the vehicle, a posted speed limit on a road, and/or a location of the vehicle, and further wherein identifying the opportune time comprises selecting a period of time when the vehicle is moving in a cruising mode of operation, is in the stopped state, or is moving in stop-and-go traffic.

9. The method of claim 8, further comprising:
evaluating, by the processor, a physiological condition of the occupant of the vehicle; and
offering, by the processor, the relaxation regimen based on evaluating the physiological condition and on identifying the opportune time.

10. The method of claim 9, wherein the physiological condition of the occupant comprises one of a heart rate, heart-rate variability, a breathing rate, and a blood oxygen level.

11. The method of claim 7, wherein the relaxation regimen comprises a breathing exercise and/or a massaging function.

12. The method of claim 11, further comprising:
operating, by the processor, an audio system, a display system and/or a haptic element to interact with the occupant during the breathing exercise.

13. The method of claim 11, further comprising:
identifying, by the processor, a first seat in which the occupant is seated; and
activating a massager system in the first seat to provide the massaging function to the occupant of the first seat.

14. The method of claim 7, wherein the occupant is a driver of the vehicle, and wherein identifying the opportune time comprises:
identifying, by the processor, over a period of time, a speed of movement of the vehicle, a stopped or parked condition of the vehicle, an acceleration characteristic of the vehicle, a braking characteristic of the vehicle, a direction of travel of the vehicle, a posted speed limit on a road, and/or a location of the vehicle.

15. A vehicle comprising:
a relaxation regimen provider system comprising:
a memory that stores computer-executable instructions; and
a processor configured to access the memory and execute the computer-executable instructions to perform operations comprising:
evaluating a physiological condition of an occupant of the vehicle;
identifying an opportune time to offer a relaxation regimen to the occupant of the vehicle; and
offering the relaxation regimen based on evaluating the physiological condition and/or on identifying the opportune time.

16. The vehicle of claim 15, wherein the physiological condition of the occupant comprises one of a heart rate, heart-rate variability, a breathing rate, and a blood oxygen level and wherein the opportune time occurs during one of when the vehicle is moving in a cruising mode of operation, is in a stopped or parked state, or is moving in stop-and-go traffic.

17. The vehicle of claim 16, further comprising:
a sensor system configured to sense the physiological condition of the occupant of the vehicle, the sensor system comprising at least one of a wearable item of clothing, a smartwatch, an activity tracker, a smartphone, a pressure transducer, a camera vision system, a radar, or a heart rate monitor.

18. The vehicle of claim 17, further comprising:
a vehicle computer configured to communicate to the relaxation regimen provider system a speed of movement of the vehicle, a stopped or parked condition of the vehicle, an acceleration characteristic of the vehicle, a braking characteristic of the vehicle, a direction of travel of the vehicle, a posted speed limit on a road, preferences of the occupant, relaxation regime usage patterns, and/or a location of the vehicle.

19. The vehicle of claim 15, wherein the processor is configured to access the memory and execute additional computer-executable instructions to perform operations comprising:
tailoring, by the processor, the relaxation regimen based on a preference of the occupant.

20. The vehicle of claim 19, wherein the relaxation regimen comprises a breathing exercise and/or a massaging function.
